(19) United States
(12) Patent Application Publication
(10) Pub. No.: US 2014/0315870 A1
(43) Pub. Date: Oct. 23, 2014

(54) COMPOSITION COMPRISING A GLUCOCORTICOID AND A THIAZOLIDINEDIONE FOR INDUCING COMPELTE ADIPOGENIC DIFFERENTIATION OF MAMMALIAN STEM CELLS

(71) Applicant: UNIVERSIDAD DEL DESARROLLO, Las Condes, Santiago (CL)

(72) Inventors: Paulette Conget Molina, Lo Barnechea (CL); David Contador Martinez, Lo Barnechea (CL)

(21) Appl. No.: 14/354,040

(22) PCT Filed: Dec. 24, 2012
(86) PCT No.: PCT/CL2012/000076

§ 371 Date: Apr. 24, 2014

(30) Foreign Application Priority Data
Oct. 24, 2011 (CL) 2642-2011

(51) Int. Cl. A61K 31/573; A61K 31/4439; C12N 5/077

(52) U.S. Cl. CPC A61K 31/573; C12N 5/0653; A61K 31/4439
USPC 514/171; 435/377; 435/366

(57) ABSTRACT

The present invention relates to the provision of a composition comprising a glucocorticoid and a thiazolidinedione for inducing adipogenic differentiation, thereby successfully generating functional stem cells, from non-differentiated embryonic or adult stem cells originating from humans or other mammals, special preference being given to human mesenchymal stem cells. The preferred glucocorticoid is dexamethasone and the preferred thiazolidinedione is rosiglitazone. However, the invention extends to the families of both compounds.

### TECHNICAL PROBLEM AND STATE OF THE ART

Adipogenesis process follows a sequence of hierarchical steps. In vitro, it has been described that prior to differentiation, adipogenic precursors must be in a quiescent state (Gregoire et al, 1998). This is necessary but not essential, since low density cultures of cells have shown that they can also initiate their differentiation. What is essential for stimulating adipogenesis in vitro is the exposure of the cells to differentiation inducers (Prokesch et al, 2009).

Currently, the most used adipogenic stimulus is one patented by Osiris Therapeutics Inc. (U.S. Pat. No. 6,322,784B1). This was originally implemented to induce differentiation of adipocytes to mesenchymal stem cells, and combines a glucocorticoid (dexamethasone), a phosphodiesterase inhibitor (isobutyl-methylxantine), a cyclooxygenase inhibitor (indometacine) and insulin. This classical cocktail has some limitations for its use, among them: i) it does not efficiently promote adipogenic differentiation of mesenchymal stem cells in some mammal species, ii) adipocytes generated using this cocktail are incompletely differentiated, iii) it must be prepared each time for its use since its stability in time is limited, iv) stock solutions of their constituents change their physicochemical properties in time (precipitation, turn turbid, etc.). Opposed, the present invention—detailed in herebelow—i) promotes adipogenic differentiation of mesenchymal stem cells of different mammal species, even those refractory to the classical cocktail, ii) allows generating functional mature adipocytes. That is, cells showing features characteristic of adipocytes found in vivo, iii) the composition is stable over the period that it would be used, iv) stock solutions do not suffer significant variations in time. Thus, the invention presents evident advantages over the classical cocktail both during use as well as commercialization.

The present invention is directed to provide a composition comprising glucocorticoid and a thiazolidinedione for inducing adipogenic differentiation, since:
Glucocorticoids play an important role both in in vitro as well as in vivo adipogenesis. In the latter case, it has been shown an association between hypercorticoidism and visceral adiposity (Berger et al., 2001). Glucocorticoid receptors are expressed in primary preadipocytes and in cell lines established from them (Joyner et al., 2000). It is known that glucocorticoids activate C/EBPdelta (CCAAT enhancer binding protein delta) (Wiper-Bergeron et al., 2003) and in turn C/EBPalpha and PPARgamma (Peroxisome Proliferator Activated Receptor gamma) both known as intracellular regulators of adipogenic differentiation (Wu et al., 1996).
PPARgamma ligands have been used as enhancers for preadipocytes to adipocytes differentiation (Prokesch et al, 2009). PPARgamma is a nuclear receptor and acts as a transcription factor when bound to its endogenous (e.g. prostaglandin J2 metabolite) or exogenous (e.g. thiazolidinedione , such as rosiglitazone, pioglitazone, troglitazone) ligands. Some thiazolidinedione s have been used as antidiabetic agents (Lehmann et al., 1995). They act by decreasing hyperglycemia and hyperinsulinemia, and increase adipose, skeletal muscle, and hepatic tissue sensibility to insulin. Recently, the United States Food and Drug Administration (FDA) suspended rosiglitazone use in humans since its chronic consumption increases in 50% the chance for cardiovascular accidents (Niseen and Wolski, 2007).

Currently available documents related to differentiation, glucocorticoids, and thiazolidinedione s are presented and discussed here below:

“Thiazolidinedione effects on glucocorticoid receptor-mediated gene transcription and differentiation in osteoblastic cells” Johnson et al., 1999: using MB1.8 cell lines (immortalized osteoblasts), SaOS-2/B10 (osteosarcoma cells) and CV-1 (kidney cells), researchers showed that only MB1.8 cells responded to thiazolidinedione, modifying their genomic response to glucocorticoids. In consequence, they concluded that the sensibility to these molecules varies from cell to cell, being not possible to predict a priori if a determined cell would respond or not to a thiazolidinedione. Thus, this work differs from the present invention in terms of its objectives: evaluating if activating PPARgamma modulates the genomic response to glucocorticoids and if this results in a phenotype change in bone cells already differentiated (osteoblasts) versus evaluating the effect of a glucocorticoid and a thiazolidinedione on adipogenic differentiation of stem cells. Also, none of the conclusions proposed by the authors allows assuming that exposition to glucocorticoid combined with a thiazolidinedione would induce adipogenic differentiation of undifferentiated stem cells. Furthermore, the table of example 5 of the present document compares in detail the description found in the referred scientific publication versus the present invention.

“Adipocyte differentiation and mitochondrial biogenesis regulating factors of antiretroviral drugs” Rodriguez, 2002 (postgraduate thesis): the author analyzes different adipogenic transcription factors and recognizes the importance of glucocorticoid and PPARgamma receptors in adipogenic differentiation. In said thesis, there is no insinuation that their ligands on their own would promote differentiation of stem cells.

“Characterization of adipocyte differentiation from human mesenchymal stem cells in bone marrow” Qian et al., 2010: presents some of the molecular mechanisms associated to adipogenic differentiation of human mesenchymal stem cells. In this scientific publication, induction to adipogenic differentiation is made with the classical cocktail. In no place in the text is made reference to the possibility of using a glucocorticoid and a thiazolidinedione for induction of adipogenic differentiation of stem cells.

“Development of a rapid culture method to induce adipocyte differentiation of human bone marrow-derived mesenchymal stem cells” Ninomiya et al., 2010: the authors propose a “new” adipogenic cocktail consisting of dexamethasone, isobutyl-methylxantine, insulin, and rosiglitazone; which promotes generation of adipocytes in shorter times (7-8 days) than the classical cocktail (2-3 weeks). In this case, rosiglitazone is used for accelerating maturation and not to induce differentiation. Also, this article does not determine the level of maturity nor the functionality of the generated adipocytes.

“DNA binding-dependent glucocorticoid receptor activity promotes adipogenesis via K\ddot{r}uppel-like factor 15 gene expression” Asada et al., 2011: specifically studies the effect of dexamethasone over the adipogenic differentiation process in two cellular models: fibroblasts derived from mouse embryos called MEF (Mouse Embryonic Fibroblasts) and human mesenchymal stem cells. The main objective of this work is determine the role of dexamethasone treatment over early events in adipogenic differentiation. To this end, dexamethasone, isobutyl-methylxantine, insulin, and pioglitazone were used as differentiation stimulus. In this document, there is no reference to the specific combination of a glucocorticoid and a thiazolidinedione for inducing adipogenic differentiation. Nor to the level of maturity or functionality of the generated adipocytes.

“Adipogenic differentiation of human mesenchymal stem cells” U.S. Pat. No. 6,322,784B1 discloses that human mesenchymal stem cells can be differentiated to adipocytes when treated with glucocorticoids, a compound that elevates intracellular levels of cAMP—either increasing the production of cAMP or inhibiting its degradation—(isobutyl-methylxantine) and insulin. In consequence, said patent proposes the use of three compounds, of which only one is shared with the present invention, to promote adipogenic differentiation. Also, no description regarding the level of maturity or functionality of the generated adipocytes is found.

“Adipogenic differentiation of human mesenchymal stem cells” U.S. Pat. No. 6,709,864B1 shows adipogenic differentiation of human mesenchymal stem cells with a glucocorticoid, a compound that increases intracellular levels of cAMP (similar to U.S. Pat. No. 6,322,784B1), insulin, and a compound that increases the expression of PPARgamma and/or increases its affinity for the DNA binding site (15-deoxy-delta-12,14-prostaglandinJ2). In consequence, said patent proposes to use a mix of four compounds, of which only one is shared with the present invention, to promote adipogenic differentiation. Also, no description regarding maturity or functionality of the generated adipocytes is found.

The prior art described herein shows solutions to adipogenic differentiation of stem cells and studies with a description of the use of glucocorticoids and thiazolidinedione to those ends. Nevertheless, none of them proposes or shows the use of them in a combination to induce adipogenic differentiation of stem cells, thus the present invention is novel. Also, the reported data is not enough for an expert in the art to find obvious that the combined use of a glucocorticoid and a thiazolidinedione would promote differentiation of stem cells to functionally mature adipocytes, thus the present invention is inventive. Lastly, in a surprising and unexpected manner, the inventors have observed that the use of the composition comprising a glucocorticoid and a thiazolidinedione robustly promotes adipogenic differentiation of stem cells that respond in a discreet manner or do not respond to the conventional stimulus (classical cocktail). Also, the adipocytes generated from stem cells exposed to the composition are mature and functional, i.e. they have completed the differentiation process and, among other features, they possess the capacity to be highly sensitive to insulin and secrete adipokines.

### FIGURES

The figures described in here below are presented with the aim of showing information supporting and exemplifying the description of the composition, therefore, they are not supposed to restrict the invention and in no way should be considered as limiting the scope of the proposed invention.

FIG. 1, the invention induces adipogenic differentiation of human mesenchymal stem cells.

Cells were exposed to a medium without stimulus (A), or a medium supplemented with DMSO (B), classical cocktail (C), the composition (D), dexamethasone (E), rosiglitazone (F), isobutyl-methylxantine+rosiglitazone (G) or indometacine+rosiglitazone (H). At 14 days, cells were dyed with Oil Red and were analyzed under light microscopy. (n=7).

FIG. 2, the invention induces adipogenic differentiation of human mesenchymal stem cells at different concentrations.

Cells were exposed to the medium supplemented with the composition comprising dexamethasone and rosiglitazone in the following concentrations (μM): 1+10 (D), 0.1+1 (Da), 0.5+5 (Db), 2+20 (Dc), 5+50 (Dd), 10+100 (De), 1+1 (Df), 1+5 (Dg), 1+20 (Dh), 1+50 (Di), 1+100, (Dj), 0.1+10 (Dk), 0.5+10 (Dl), 2+10 (Dm), 5+10 (Dn), 10+10 (Do), 0.1+100 (Dp), 0.5+50 (Dq), 1+20 (Dr), 2+10 (Ds), 5+5 (Dt), or 10+1 (Du). At 14 days, cells were dyed with Oil Red and analyzed under light microscopy to determine the presence of adipocytes in each sample. (n=2).

FIG. 3, the invention significantly induces adipogenic differentiation of human mesenchymal stem cells at different concentrations.

Cells were exposed to the medium supplemented with the composition comprising dexamethasone and rosiglitazone in the following concentrations (μM): 1+10 (D), 0.1+1 (Da), 0.5+5 (Db), 2+20 (Dc), 5+50 (Dd), 10+100 (De), 1+1 (Df), 1+5 (Dg), 1+20 (Dh), 1+50 (Di), 1+100, (Dj), 0.1+10 (Dk), 0.5+10 (Dl), 2+10 (Dm), 5+10 (Dn), 10+10 (Do), 0.1+100 (Dp), 0.5+50 (Dq), 1+20 (Dr), 2+10 (Ds), 5+5 (Dt), or 10+1 (Du). At 14 days, cells were dyed with Nile Red and analyzed with flow cytometry to determine the percentage of adipocytes in each sample. (n=1).

FIG. 4, the invention induces adipogenic differentiation of mesenchymal stem cells from other mammals.

Cells obtained from mouse (I), rat (II), rabbit (III), or dog (IV), were exposed to a medium without stimulus (A), or a medium supplemented with DMSO (B), classical cocktail (C), the composition (D). At 14 days, cells were dyed with Oil Red and analyzed under light microscopy to determine the presence of adipocytes in each sample. (n=3).

FIG. 5, the invention significantly induces adipogenic differentiation of mouse mesenchymal stem cells.

Cells obtained from mouse (I) were exposed to a medium without stimulus (A), or a medium supplemented with DMSO (B), classical cocktail (C), the composition (D). At 14 days, cells were trypsinized, dyed with Nile Red and analyzed with flow cytometry to determine the percentage of adipocytes in each sample. Data correspond to average±standard error. (n=3).

FIG. 6, the invention significantly induces adipogenic differentiation of rat mesenchymal stem cells.

Cells obtained from rat (II) were exposed to a medium without stimulus (A), or a medium supplemented with DMSO (B), classical cocktail (C), the composition (D). At 14 days, cells were trypsinized, dyed with Nile Red and analyzed with flow cytometry to determine the percentage of adipocytes in each sample. Data correspond to average±standard error. (n=3).

FIG. 7, the invention significantly induces adipogenic differentiation of rabbit mesenchymal stem cells.

Cells obtained from rabbit (III) were exposed to a medium without stimulus (A), or a medium supplemented with DMSO (B), classical cocktail (C), the composition (D). At 14 days, cells were trypsinized, dyed with Nile Red and analyzed with flow cytometry to determine the percentage of adipocytes in each sample. Data correspond to average±standard error. (n=3).

FIG. 8, the invention significantly induces adipogenic differentiation of dog mesenchymal stem cells.

Cells obtained from dog (IV) were exposed to a medium without stimulus (A), or a medium supplemented with DMSO (B), classical cocktail (C), the composition (D). At 14 days, cells were trypsinized, dyed with Nile Red and analyzed with flow cytometry to determine the percentage of adipocytes in each sample. Data correspond to average±standard error. (n=3).

FIG. 9, adipocytes generated from human mesenchymal stem cells exposed to the invention are insulin-sensitive.

Cells were exposed to medium supplemented with the composition (D). At 14 days, cells were stimulated with 0.1; 1 or 10 nM insulin and then, incorporated [³H]2-DG (pmole/mg of protein) was quantified. The segmented line represents the basal level—without insulin—of [³H]2-DG. Data correspond to average ±standard error. (n=3).

FIG. 10, adipocytes generated from human mesenchymal stem cells exposed to the classical cocktail are not sensitive to insulin.

Cells were exposed to medium supplemented with the classical cocktail (C). At 14 days, cells were stimulated with 0.1; 1 or 10 nM insulin and then, incorporated [³H]2-DG (pmole/mg of protein) was quantified. The segmented line represents the basal level—without insulin—of [³H]2-DG. Data correspond to average±standard error. (n=3).

FIG. 11, adipocytes generated from human mesenchymal stem cells exposed to the invention express LEPTIN gene.

Cells were exposed to a medium without stimulus (A), or a medium supplemented with DMSO (B), classical cocktail (C), the composition (D). At 14 days, RNA was isolated from cells for further quantification of LEPTIN and GAPDH mRNA through real time RT-PCT (relative abundance expressed in arbitrary units). Data correspond to average±standard error. (n=7).

FIG. 12, adipocytes generated from human mesenchymal stem cells exposed to the invention secrete leptin.

Cells were exposed to a medium without stimulus (A), or a medium supplemented with DMSO (B), classical cocktail (C), the composition (D). At 14 days, conditioned medium was collected for further quantification of leptin protein levels (pg(mL) through ELISA. Data correspond to average±standard error. (n=7).

### THE INVENTION

The present invention is directed to a composition to induce adipogenic differentiation of undifferentiated stem cells to a mature functional adipocyte state. This reactive consists in the combination of two synthetic compounds: a glucocorticoid and a thiazolidinedione in a biocompatible vehicle, with no need for adding additional components.

Empirical data supporting this patent application were obtained with one representative of each family (glucocorticoid: dexamethasone and thiazolidinedione: rosiglitazone). Nevertheless, it is extensive to the families of compounds and not only to the representatives of each of them.

Stem cells used in the present invention can come from different mammal species, not only human.

Cell type to use in the present invention corresponds to all kinds of stem cells, either adult or embryonic, among which mesenchymal stem cells are preferred.

According to the merits of the present invention, the composition can be produced at industrial level and can be commercialized as a reactive to be used for the following ends:

1. Stem cell characterization.

2. Cellular and molecular mechanism of adipogenic differentiation studies.

3. ex vivo (out of the patient) generation of adipocytes from stem cells. These adipocytes could be used as a source of molecules of biological interest (adipokines, cytokines, etc).

4. ex vivo (out of the patient) generation of adipocytes from stem cells. These adipocytes could be used for transplanting to the same patient (autologous) or to another (allogeneic).

5. in vivo (in the patient) generation of adipocytes from stem cells, for example in metabolic diseases wherein instead of a transplant of adipose tissue, adipocytes, or undifferentiated stem cells, the composition can be administered.

6. in vivo (in the patient) generation of adipocytes from stem cells, for example in reconstructive surgeries wherein instead of transplanting adipose tissue, adipocytes, or undifferentiated stem cells, the composition can be administered.

### EXAMPLES

The examples described in here below are accompanied only for illustrative purposes in order to facilitate the understanding of the set of claims and are not intended to limit in any manner the scope of the requested claims.

### Example 1

### Induction to Adipogenic Differentiation of Human Mesenchymal Stem Cells

Human mesenchymal stem cells were seeded at a density of 2.5·10⁴ cells/cm² in minimal essential medium alpha supplemented with fetal bovine serum 10% (v/v)+gentamycin 80 μg/ml (alpha-10 from here) and were kept under humid atmosphere air/CO₂ 95/5% at 37° C. The next day, the medium was replaced with alpha-10 supplemented with dexamethasone 1 mM+isobutyl-methylxantine 100 mg/mL+indometacine 100 mM+insulin 0.2 IU/mL (classical cocktail) or dexamethasone 1 μM+rosiglitazone 10 μM (composition). At 14 days, adipogenic differentiation was evaluated, by dying with Oil Red or Nile Red. To this, cells were washed three times with PBS and incubated in an aqueous solution of isopropanol 60% (v/v) saturated with Oil Red O for one hour at room temperature. Then, cells were washed three times with PBS and were observed under inverted contrast phase light microscope. Digital photographic record was left with the results. For Nile Red dying and flow cytometry analysis, cells were tripsynized, resuspended in alpha-10 and maintained in suspension for 30 minutes at room temperature. Then, Nile Red 1 mg/mL was added in DMSO and cells were incubated for 5 minutes. Cells were acquired in a DAKO Cyan cytometer. Adipocytes were identified in function of the following three criteria: size (FSC), granularity or internal complexity (SSC), and fluorescence intensity in channel FI1 (Nile Red) which were previously set in the device. A total of 10,000 events were analyzed for each sample.

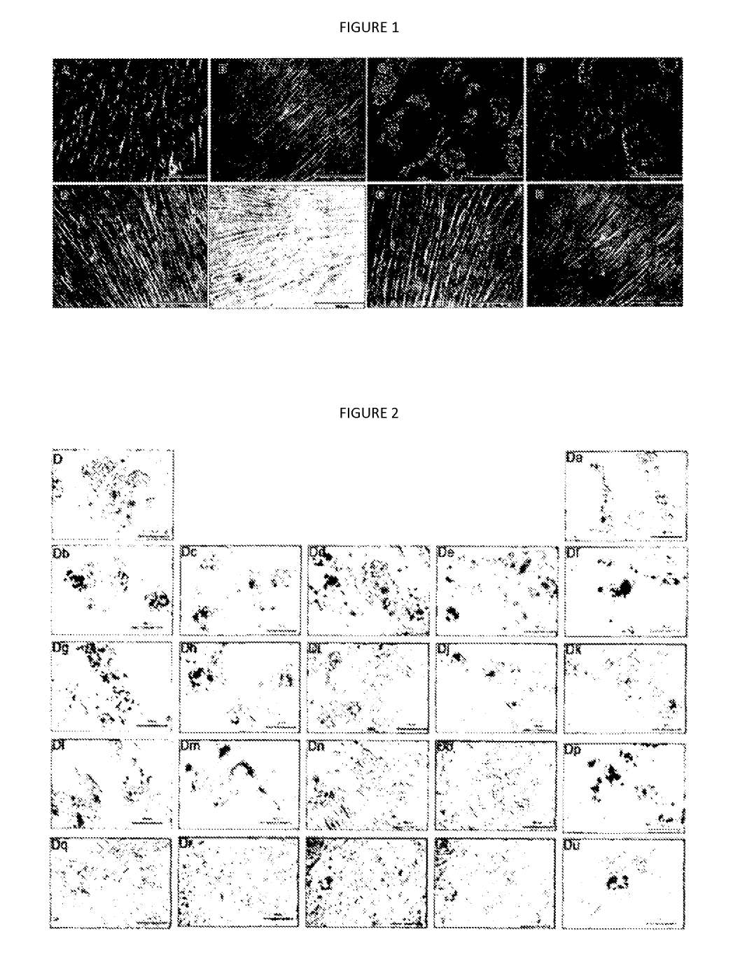

After exposing the human mesenchymal stem cells for 14 days to the composition, appearance of cells that inside them had neutral lipids drops, characteristic of adipocytes (FIG. 1). Opposed, cells incubated with each of the components separately (dexamethasone or rosiglitazone) or one of them plus one of the components of the classical cocktail (isobutyl-methylxantine+rosiglitazone, indometacine+rosiglitazone) were not differentiated. Therefore, it is demonstrated that the invention, and not any combination of molecules, promotes adipogenic differentiation of stem cells.

### Example 2

### Adipogenic Differentiation Induction of Human Mesenchymal Stem Cells Using Different Concentrations of Dexamethasone+rosiglitazone.

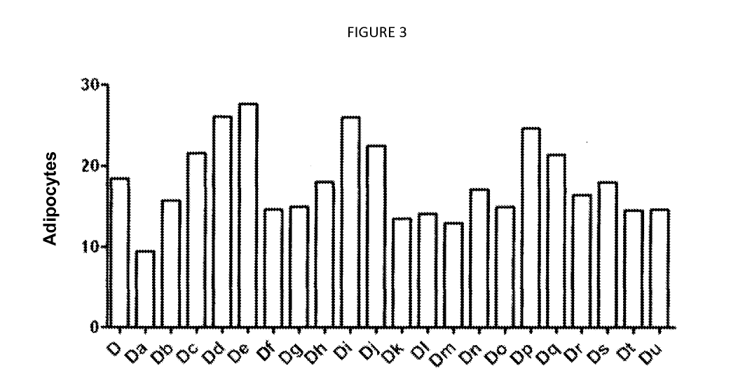

In similar conditions to those of Example 1, it was evaluated if the composition promoted human mesenchymal stem cell differentiation when dexamethasone is present in a range comprised between 0.1 and 10 μM, and rosiglitazone present in a range comprised between 1 and 100 μM. All evaluated combinations induced adipogenic differentiation of undifferentiated stem cells (FIGS. 2 and 3). Thus, it is demonstrated that the invention is effective in a wide range of concentrations.

### Example 3

### Induction of Adipogenic Differentiation of Mesenchymal Stem Cells from Other Mammals.

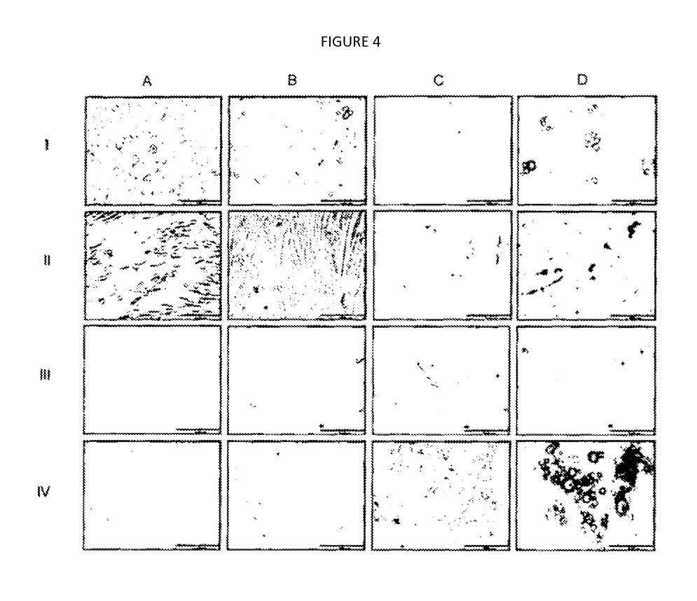

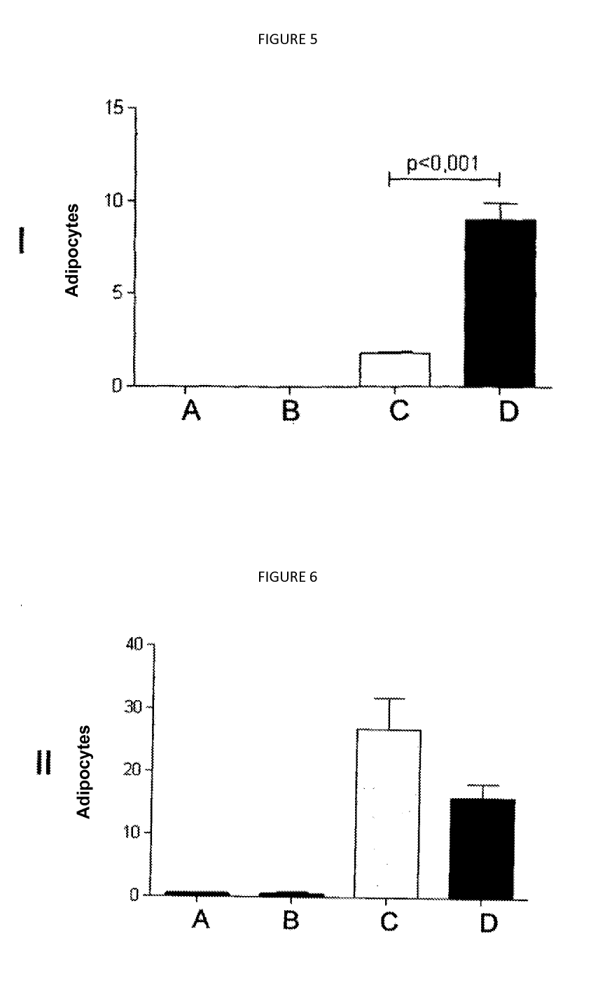

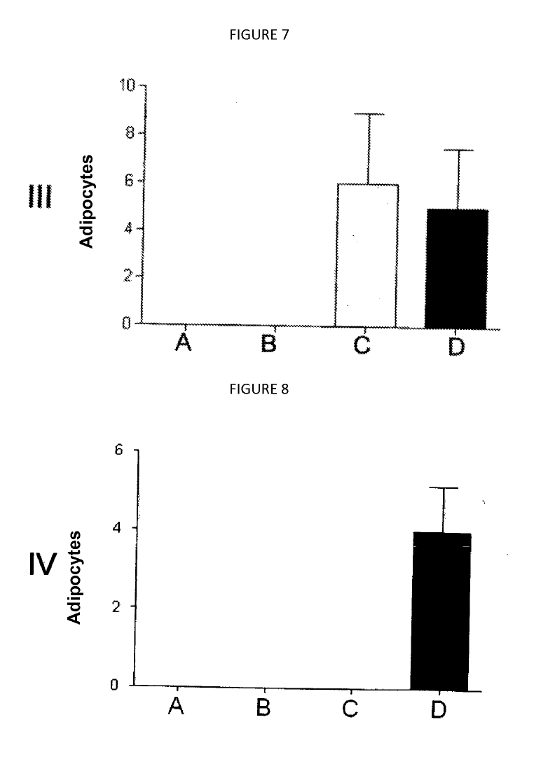

In similar conditions to the ones described in Example 1, it was evaluated if the composition promoted differentiation of mesenchymal stem cells derived from other mammals, in particular, from mouse, rat, rabbit, and dog. The classical cocktail and the composition induced, in a similar magnitude, differentiation of rat and rabbit stem cells (FIGS. 4, 6, and 7). On the other hand, while the classical cocktail marginally induced adipogenic differentiation of mouse and dog stem cells, the composition promoted them efficiently (FIGS. 4, 5, and 8). These results allow concluding that the invention has broader applications than the classical cocktail. Thus, the composition is the first stimulus of adipogenic differentiation that works in multiple mammal species.

### Example 4

### Generation of Functional Mature Adipocytes

All the differentiation process comprises two steps, this is commitment and maturation. A cell is functional when it completes its maturation process. In the case of adipocytes, when besides accumulation of triacylglycerides drops and expression of genes characteristic of said lineage, it loses its proliferative potential, is sensitive to insulin, and secretes adipokines (e.g. leptin) (Pelleymounter et al., 1995; Friedman and Halaas, 1998). For determining the maturity level of adipocytes generated with the composition, its sensibility to insulin, and its capacity to express and secrete leptin were evaluated.

Insulin Sensibility: human mesenchymal stem cells were seeded at a density of 2.5·10⁴ cells/cm² and incubated for 14 days in presence of the classical cocktail or the composition. Sensibility of these cells to insulin was evaluated in function of incorporation of a radioactive analog of glucose [³H]2-deoxyglucose ([³H]2-DG). Prior to determination, cells were deprived of fetal bovine serum for 4 hours. Then, they were incubated with insulin in increasing concentrations for 30 minutes, washed with PBS at 37° C., and incubated in PBS containing 2-DG 4 mM and [³H]2-DG 2 μCi/mL for 1 minute. Cells were washed with PBS at 4° C. and frozen at −20° C. After lysing with formic acid 0.5N for 1 hour, an aliquot was sampled for quantification of proteins and the remaining was incubated in pure formic acid for 30 minutes. Finally, the radioactivity level that was incorporated in the cells was determined with a scintillation counter LKB Rackbeta 1217.

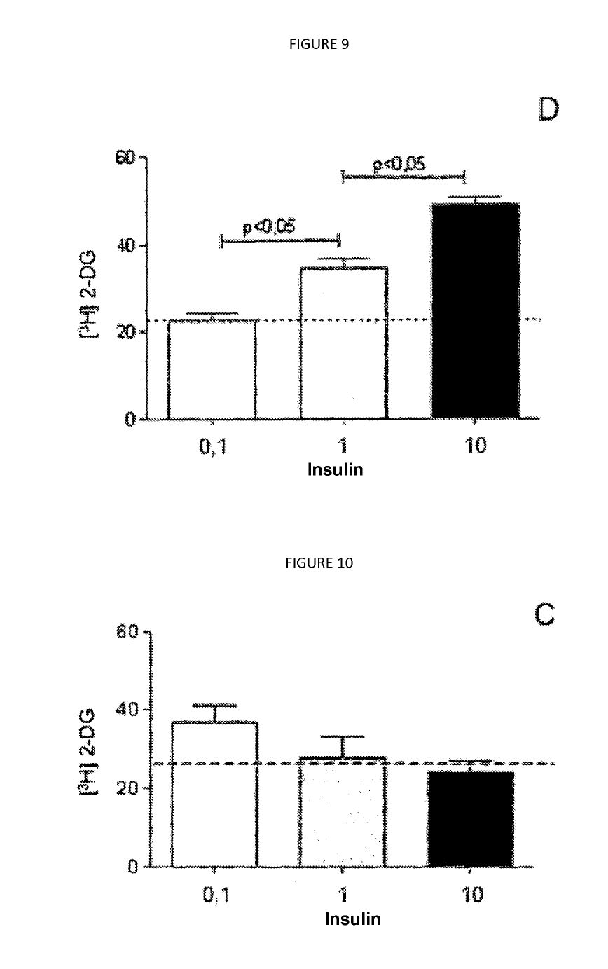

Adipocytes generated from human mesenchymal stem cells exposed to the composition are insulin-sensitive (FIG. 9), opposed to those generated using the classical cocktail, that are not (FIG. 10).

Leptin Expression: once the human mesenchymal stem cells reached the incubation time with the adipogenic inducers, RNA was extracted. Cells were washed with PBS, then were homogenized with 1 mL Trizol®. Chloroform was added to the homogenate, tubes were vigorously agitated, incubated 2 minutes at room temperature and centrifuged at 13,000×g at 4° C. for 15 minutes. Afterwards, supernatant was discarded and the precipitate was washed with 1 mL of an ethanol solution 75% (v/v) prepared with nuclease free water, centrifuged at 12,000×g at 4° C. for 10 minutes, eliminating the supernatant and resuspending the pellet in 20 μL of nuclease free water, isolated RNA was stored at −80° C. for later analysis.

PCR reaction was performed in a LightCycler® thermocycler, using the FastStar DNA Master SYBR Green I LightCycler® kit. Each reaction mix (10 μL) contained: 50 ng cDNA; each primer 0.5 mM and MgCl₂ 3mM. Data were quantified through ΔCt method (Schmittgen and Livak, 2008).

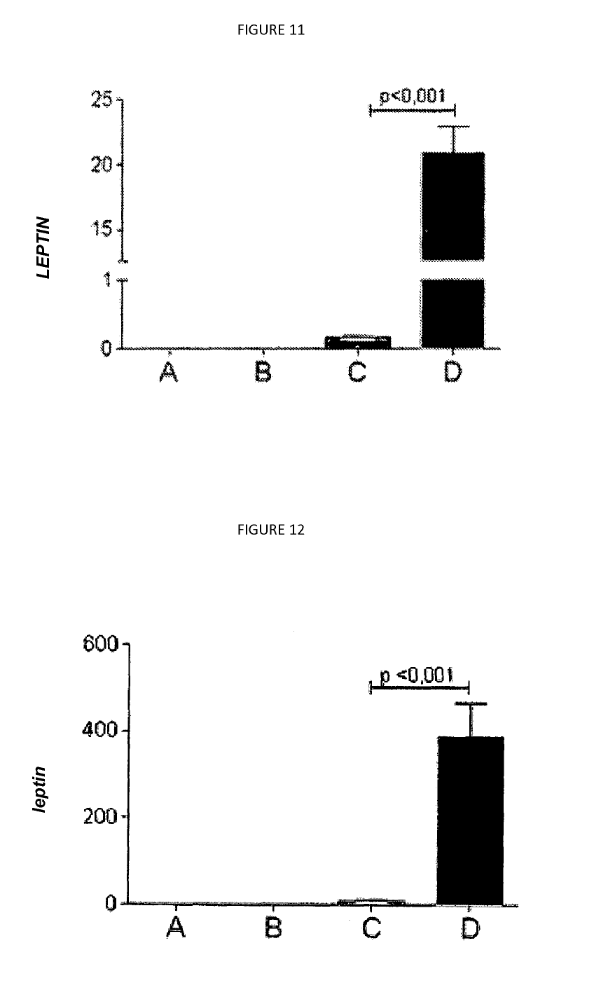

As shown in FIG. 11, while adipocytes generated from human mesenchymal stem cells using the composition express LEPTIN gene, those generated using the classical cocktail do not.

Leptin Secretion: the culture medium was replaced with fetal bovine serum free medium. At 24 hours, the conditioned medium was collected and centrifuged at 2,500×g for 10 minutes. Then, the supernatant was recovered and stored in a clean tube at −80° C. For leptin quantification, ELISA Quantikine® human leptin immunoassay from R&D Systems was used. The percentage of adipocytes using Nile Red and flow cytometry, from cultures from where the conditioned medium was collected, was also determined.

As shown in FIG. 12, adipocytes generated with the composition, release leptin, whereas those generated with the classical cocktail do not.

Taken together, these results show that the invention allows to generate mature functional adipocytes, which the classical cocktail does not.

### Example 5

Comparative analysis between the results shown in the closest document in the prior art and the present invention.

The following Table compares the description found in Johnson et al, 1999 versus the present invention.

| | Johnson et al., 1999 | | Present invention | |
|---|---|---|---|---|
| Objective | studying effect of different | | induce differentiation of | |
| | PPARs ligands on expression of | | mesenchymal stem cells to | |
| | genes induced by | | mature functional adipocytes | |
| | glucocorticoids- osteoblast | | | |
| | phenotype | | | |
| Cell types | osteoblasts (differentiated cells) | | mesenchymal stem cells | |
| | | | (undifferentiated cells) | |
| Cell source | calvareal mouse (MB1.8) human | | mouse, rat, rabbit, dog, and | |
| | osteosarcoma (SaOS-2) | | human bone marrow | |
| | monkey renal epithelial cells | | (primary cultures) | |
| | (CV-1) (immortalized cell lines) | | | |
| Glucocorticoid | dexamethasone (nM) | | dexamethasone (μM) | |
| Thiazolidinedione | rosiglitazone (μM) | | rosiglitazone (μM) | |
| Exposure time | 48 hours | | 14 days | |
| Outcomes | i) | expression of genes induced | i) | appearance of neutral fat |
| | | by glucocorticoids | | drops |
| | ii) | expression of two osteoblast | ii) | characteristic adipocyte |
| | | genes | | gene expression |
| | iii) | activity of an osteoblast | iii) | insulin sensitivity |
| | | enzyme | iv) | adipokine secretion |
| | iv) | mineralization capacity in | | |
| | | vitro | | |
| | v) | expression of an adipocyte | | |
| | | gene | | |
| Results | i) | regarding gene expression | Exposition to the composition | |
| | | induced by glucocorticoids: | and not to its separated or | |
| | | in osteoblasts, rosiglitazone | sequential components, results | |
| | | increases | in: | |
| | | kidney epithelial cells, | i) | appearance of neutral fat |
| | | rosiglitazone does not affect | | drops |
| | ii) | regarding the osteoblast | ii) | induction of expression of |
| | | phenotype to exposition to | | genes characteristics of |
| | | dexamethasone and | | adipocytes |
| | | rosiglitazone: | iii) | insulin sensitivity |
| | | decreases expression of | iv) | induction of secretion of |
| | | genes and activity of an | | adipokines |
| | | enzyme characteristics of | | |
| | | osteoblasts | | |
| | | does not significantly affect | | |
| | | capacity to mineralize in vitro | | |
| | | increases expression of a | | |
| | | gene characteristic of | | |
| | | adipocytes | | |

| | | | |
|---|---|---|---|
| Conclusion | i) | exposition to rosiglitazone | the composition induces |
| | | increases expression of | differentiation of mesenchymal |
| | | genes induced by | stem cells, from different |
| | | glucocorticoids | species, to mature functional |
| | ii) | rosiglitazone slightly alters | adipocytes. |

the phenotype of
osteoblasts exposed to
dexamethasone

### BIBLIOGRAPHIC REFERENCES

Asada M, Rauch A, Shimizu H, Maruyama H, Miyaki S, Shibamori M, Kawasome H, Ishiyama H, Tuckermann J, Asahara H. DNA binding-dependent glucocorticoid receptor activity promotes adipogenesis via Krüppel-like factor 15 gene expression. Lab Invest. (2011) 91:203-15.

Berger J, Tanen M, Elbrecht A, Hermanowski-Vosatka A, Moller D E, Wright S D, Thieringer R. Peroxisome proliferator-activated-gamma ligands inhibit adipocyte 1beta-hydroxysteroid dehydrogenase type 1 expression and activity. J Biol Chem. (2001) 276:12629-35.

Gregoire F M, Smas C M, Sul H S. Understanding adipocyte differentiation. Physiol Rev. (1998) 78:783-809.

Johnson T, Vogel R, Rutledge S J, Rodan G, Schmidt A. Thiazolidinedione Effects on Glucocorticoid Receptor-Mediated Gene Transcription and Differentiation in Osteoblastic Cells. Endocrinology (1999) 140:3245-54.

Joyner J M, Hutley L J, Cameron D P. Glucocorticoid receptors in human preadipocytes: regional and gender differences. J Endocrinol. (2000)166:145-52.

Lehmann J M, Moore L B, Smith-Oliver T A, Wilkison W O, Willson T M, Kliewer S A. An antidiabetic thiazolidinedione is a high affinity ligand for peroxisome proliferator-activated receptor gamma (PPAR gamma). J Biol Chem. (1995) 270:12953-6.

Ninomiya Y, Sugahara-Yamashita Y, Nakachi Y, Tokuzawa Y, Okazaki Y, Nishiyama M. Development of a rapid culture method to induce adipocyte differentiation of human bone marrow-derived mesenchymal stem cells. Biochem Biophys Res Commun. (2010) 394:303-8.

Niseen S E and Wolski K. Effect of rosiglitazone on the risk of myocardial infarction and death from cardiovascular causes. N Engl J Med. (2007) 356:2457-71.

Pelleymounter M A, Cullen M J, Baker M B, Hecht R, Winters D, Boone T, Collins F. Effects of the obese gene product on body weight regulation in ob/ob mice. Science (1995) 269: 540-3.

Prokesch A, Hackl H, Hakim-Weber R, Bornstein S R, Trajanoski Z. Novel insights into adipogenesis from omics data Curr Med Chem. (2009) 16:2952-64.

Qian S W, Li X, Zhang Y Y, Huang H Y, Liu Y, Sun X, Tang Q Q. Characterization of adipocyte differentiation from human mesenchymal stem cells in bone marrow. BMC Dev Biol. (2010) 10:47.

Rodriguez M L. (2002) Diferenciación adipocitaria y factores reguladores de la biogenesis mitocondrial de los fármacos antirretrovirales. Programa de Doctorado de Biomedicina, Bienio 2000-2002.

Schmittgen T D, Livak K J. Analyzing real-time PCR data by the comparative C(T) method. Nat Protoc. (2008) 3:1101-8.

Wiper-Bergeron N, Wu D, Pope I, Schild-Poulter C, Hache R J. Stimulation of preadipocyte differentiation by steroid through targeting of a HDAC1 complex. EMBO J. (2003) 22:2135-45.

Wu T J, Silverman A J, Gibson M J. FOS expression in grafted gonadotropin-releasing hormone neurons in hypogonadal mouse: mating and steroid induction. J Neurobiol. (1996) 31 :67-76.

U.S. Pat. No. 6,322,784B1. Title: ADIPOGENIC DIFFERENTIATION OF HUMAN MESENCHYMAL STEM CELLS. Assignee/Applicant: Osiris Therapeutics Inc., Baltimore.Md. Inventor: Pittenger, Mark F., Beck, Stephen C. Priority Date: 1998-10-26. Publication Date: 2001-11-27

U.S. Pat. No. 6,709,864B1. Title: ADIPOGENIC DIFFERENTIATION OF HUMAN MESENCHYMAL STEM CELLS. Assignee/Applicant: Osiris Therapeutics Inc., Baltimore.Md. Inventor: Pittenger, Mark F. | Beck, Stephen C. Priority Date: 2000-03-28. Publication Date: 2004-03-23

US20090181456A1. Title: ADIPOSE-DERIVED STEM CELLS AND LATTICES. Assignee/Applicant: Inventor: Hedrick, Marc H.; Katz, Adam J.; Lluil, Ramón; Futrell, J. William; Benhaim, Prosper; Lorenz, Hermann Peter; Zhu, Min. Priority Date: 2008-12-08. Publication Date: 2009-07-16

US20070128727A1. Title: METHODS FOR DIFFERENTIATION OF EMBRYONIC STEM CELLS. Inventor: Kraemer, Fredric, B., Shen, Wen Jun, Chen, Theresa, L. Application Date: 2006-11-06. Priority Date: 2006-11-06. Publication Date: 2007-06-07

WO2006130690A2. Title: METHODS AND COMPOSITIONS FOR INDUCING BROWN ADIPOGENESIS. Assignee/Applicant: JOSLIN DIABETES CENTER INC., US. Inventor: TSENG, Yu Hua. Priority Date: 2006-06-01. Publication Date: 2006-12-07

WO2000042211A1. Title: METHODS OF SCREENING PROTEASE INHIBITORS, OF INDUCING MICE SUSCEPTIBLE TO HIV PROTEASE INHIBITOR-INDUCED DYSLIPIDEMIA, AND GENES ASSOCIATED THEREWITH. Assignee/Applicant: GLAXO GROUP LIMITED, GB. Inventor: LENHARD, James, Martin. Priority Date: 2000-01-19. Publication Date: 2000-07-20

JP2008194044A. Title: ENDOCRINE PANCREAS DIFFERENTIATION OF ADIPOSE TISSUE-DERIVED STROMAL CELLS AND USES THEREOF. Assignee/Applicant: ARTECEL SCIENCES INC. Inventor: CHEATHAM BENTLEY, HALVORSEN YUAN-DI C, GIMBLE JEFFREY M. Priority Date: 2008-02-14. Publication Date: 2008-08-28

1. Composition based on synthetic reactants for inducing complete adipogenic differentiation, wherein the composition comprises a glucocorticoid and a thiazolidinedione in a biocompatible vehicle.

2. Composition according to claim 1, wherein the glucocorticoid is present in concentrations of around between 0.1 and 10 μM, and thiazolidinedione is present in concentrations of around between 1 and 100 μM.

3. Composition according to claim 1, wherein the biocompatible vehicle is water, ethanol, physiological serum, culture medium, chemical excipients.

4. Composition according to claim 1, wherein the glucocorticoid is dexamethasone and the thiazolidinedione is selected from troglitazone, pioglitazone, and rosiglitazone.

5. Use of a composition formed by a glucocorticoid and a thiazolidinedione for inducing adipogenic differentiation of stem cells.

6. Use according to claim 5, wherein the stem cells are mammal embryonic or adult undifferentiated stem cells.

7. Use according to claim 6, wherein the stem cells are human embryonic or adult undifferentiated cells.

8. Use according to claim 7, wherein the stem cells are human mesenchymal stem cells.

9. User according to claim 5, wherein the glucocorticoid is dexamethasone.

10. Use according to claim 9, wherein the glucocorticoid is dexamethasone in concentrations of around between 0.1 and 10 μM.

11. Use according to claim 10, wherein the glucocorticoid is dexamethasone in concentrations preferably of around between 0.5 and 2 μM.

12. Use according to claim 5, wherein the thiazolidinedione is selected among troglitazone, pioglitazone, and rosiglitazone.

13. Use according to claim 12, wherein thiazolidinedione is rosiglitazone.

14. Use according to claim 13, wherein thiazolidinedione is rosiglitazone in concentrations of around between 1 and 100 μM.

15. Use according to claim 14, wherein thiazolidinedione is rosiglitazone in concentrations of around between 5 and 20 μM.

16. Use according to claim 5, wherein the composition is for characterization of stem cells.

17. Use according to claim 5, wherein the composition is for studying cellular and molecular mechanisms of adipogenic differentiation.

18. Use according to claim 5, wherein the composition is for ex vivo generation of adipocytes from stem cells.

19. Use according to claim 5, wherein the composition is for ex vivo generation of adipocytes from stem cells to be used as source of molecules with biological interest (adipokines, cytokines, etc.)

20. Use according to claim 18, wherein the composition is for ex vivo generation of adipocytes from stem cells for transplant to the same individual (autologous) or to another one (allogeneic).

21. Use according to claim 5, wherein the composition is for in vivo generation of adipocytes from stem cells.

22. Use according to claim 21, wherein the composition is for, among others, metabolic diseases through the administration of the composition.

23. Use according to claim 22, wherein the metabolic disease is metabolic syndrome or prediabetic status.

24. Use according to claim 21, wherein the composition is for, among others, reconstructive surgeries through administration of the composition.

25. Use according to claim 24, wherein the reconstructive surgery is mammoplasty.
